(19) United States
(12) Patent Application Publication
(10) Pub. No.: US 2008/0021093 A1
(43) Pub. Date: Jan. 24, 2008

(54) Oxidation process with enhanced safety useful in the manufacture of Moxidectin

(75) Inventors: Stefania Sapienza, Catania (IT); Pasquale Massara, Reggio Calabria (IT); Marco Caraco, Siracusa (IT); Giuseppe Miraglia, Ficarazzi-Acicastello (Catania) (IT); Jignesh Patel, Plainsboro, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/821,225

(22) Filed: Jun. 21, 2007

Related U.S. Application Data
(60) Provisional application No. 60/815,725, filed on Jun. 22, 2006.

(51) Int. Cl. C07D 493/22; A01N 43/90; A61P 33/00

(52) U.S. Cl. 514/450; 549/264

(57) ABSTRACT

The present invention provides a safe and effective process for the selective oxidation of a 5-O-protected-LLF-28249-α compound to the corresponding 23-keto compound of formula I 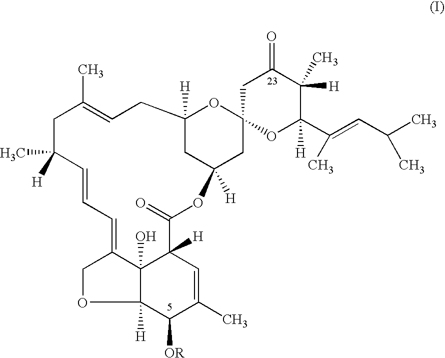 wherein R is a protecting group. The present invention also provides the use of the selective oxidation process in the manufacture of moxidectin.

This application claims the benefit under 35 U.S.C. §119(e) to co-pending U.S. provisional application No. 60/815,725, filed Jun. 22, 2006, which is hereby incorporated by reference in its entirety.

### BACKGROUND OF THE INVENTION

Moxidectin (23-methoxime-LL-F-28249-α) is a potent endectocidal agent. An important step in the manufacture of moxidectin is the oxidation of the 5-O-protected-LLF-28249-α intermediate compound. Oxidizing agents which may be used in this manufacturing step are disclosed in U.S. Pat. No. 4,988,824 and U.S. Pat. No. 6,762,327. In many instances, on a manufacturing scale, these oxidizing agents require large amounts of pyridine and a corrosive catalyst, such as dichloroacetic acid, or involve oxidizing agents, which on a manufacturing scale, may introduce unwanted risks. Further, as with all manufacturing processes, improvements in energy efficiency, in product yield and product purity are highly desirable.

Therefore, it is an object of this invention to provide an improved oxidation process for the production of moxidectin.

It is another object of this invention to provide an oxidation process, which affords mild reaction conditions and high product yields.

It is a feature of this invention that the oxidation process may utilize an oxidizing agent with enhanced safety.

These and other objects and features of the invention will become more apparent from the detailed description set forth hereinbelow.

### SUMMARY OF THE INVENTION

The present invention provides an improved process for the selective oxidation of a 5-O-protected-LLF-28249-α compound of formula II 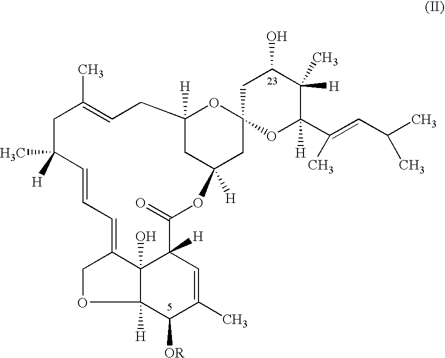
wherein R is a protecting group to the corresponding 23-keto compound of formula I 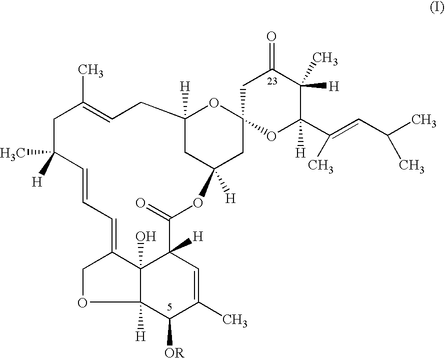
wherein R is as described for formula II which process comprises reacting said formula II compound with stabilised o-iodoxybenzoic acid, optionally in the presence of a solvent.

Also provided is the use of the improved oxidation process in the manufacture of moxidectin.

### DETAILED DESCRIPTION OF THE INVENTION

Moxidectin is a potent broad-spectrum endectocide of the macrocyclic lactone antimicrobial class. The unique activity of moxidectin against endo- and ectoparasites in both humans and animals, along with its high margin of safety, has had a tremendous impact on the control of internal and external parasites in companion animals and livestock. Therefore, availability of this compound is highly desired. Moxidectin is the 23-oxime derivative of LL-F28249-α. A process for the manufacture of moxidectin from LL-F28249-α is disclosed in U.S. Pat. No. 4,988,824 Said process includes an oxidation step wherein the oxidizing agents disclosed are conventional agents such as pyridinium dichromate, aluminum t-butoxide, o-benzoquinone, phophorous pentoxide, dicyclohexylcarbodiimide, manganese dioxide, acetic anhydride, dimethyl sulfoxide and the like or mixtures thereof. Another process, disclosed in U.S. Pat. No. 6,762,327, uses a periodinane derivative. Some common difficulties encountered in using these reagents, such as long reaction times, difficult workup procedures, possible use of a large excess of the oxidizing agent, potential instability of oxidizing agent and the like, can be problematic on a commercial manufacturing scale.

Surprisingly, it has now been found that stabilised o-iodoxybenzoic acid may be used to selectively oxidize a 5-O-protected-LL-F28249-α compound to the corresponding 5-O-protected-23-ketone compound under mild reaction conditions, with high product yield and without the hazardous chemical properties generally associated with conventional oxidizing agents.

Accordingly, the present invention provides an improved process for the selective oxidation of a 5-O-protected-LLF-28249-α compound of formula II 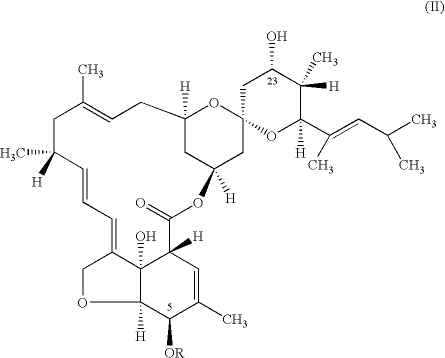
wherein R is a protecting group to the corresponding 23-keto compound of formula I 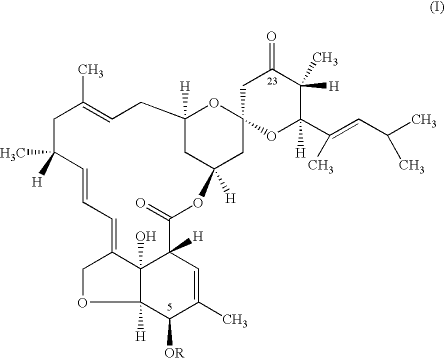
wherein R is as described for formula II which process comprises reacting said formula II compound with stabilised o-iodoxybenzoic acid, optionally in the presence of a solvent. The reaction is shown in flow diagram I wherein R represents a protecting group.

### Flow Diagram I

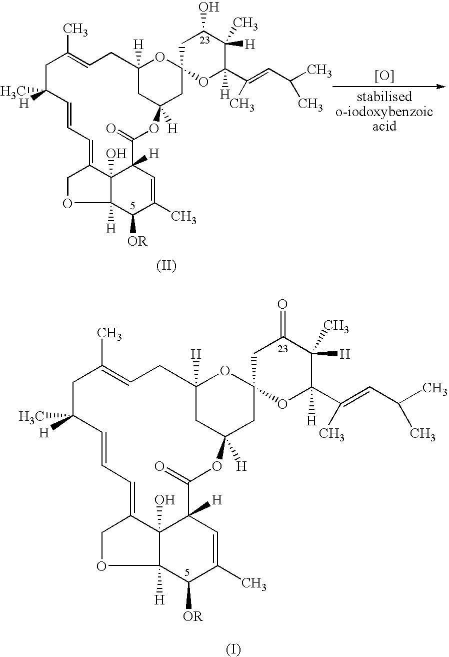

As used in the specification and claims the term “stabilised o-iodoxybenzoic acid” designates a mixture comprising about 48-50%, preferably 49%, of o-iodoxy-benzoic acid, about 28-30%, preferably 29%, of isophthalic acid and about 21-23%, preferably 22% of benzoic acid.

Solvents suitable for use in the inventive process include toluene, dimethyl sulfoxide, N-methylpyrrolidinone, or the like, or a mixture thereof, preferably toluene.

As used in the specification and claims, the term protecting group designates p-nitrobenzoyl, acetyl, benzyl, methyl, methoxymethyl, methylthiomethyl, (phenyidi-methylsilyl)methoxymethyl, p-methoxybenzyloxymethyl, o-nitrobenzylmethyl, o-nitrobenzyl-oxymethyl, 4-methoxyphenoxymethyl, guaiacolmethyl, t-butoxymethyl, 4-pentenyloxymethyl, siloxymethyl, 2-ethoxyethoxymethyl, 2,2,2-trichloroethxymethyl, 2-(trimethylsilyl)ethoxymethyl, trimethylsilyl, t-butyldimethylsilyl, phenyldimethylsilyl, or any protecting group known to protect an hydroxy group in organic synthesis, preferably p-nitrobenzoyl.

In actual practice, the stabilised o-iodoxybenzoic acid agent is admixed with a compound of formula II in a ratio of about 1.1 to 1.5 wt/wt, o-iodoxybenzoic acid to the compound of formula II, optionally in the presence of a solvent, at a temperature range of about 20° C. to 70° C., until oxidation is complete. Reaction times for the process of the invention may vary according to the amount of stabilised o-iodoxybenzoic acid agent used, the concentration of the formula II compound, the reaction temperature, or the like, in general reaction times of one to two hours are sufficient. For optimum product yield, a ratio of about 1.1 to 1.5 wt/wt of o-iodoxy-benzoic acid to the compound of formula II is suitable for use in the inventive process.

Advantageously, the process of the invention may be used in the manufacture of moxidectin. Accordingly, the present invention provides an improved process for the manufacture of moxidectin which comprises the following steps:
1) protecting the 5-hydroxy group of LL-F28249-α to give the compound of formula II;
2) reacting said formula II compound with stabilised o-iodoxybenzoic acid optionally in the presence of a solvent to give the ketone of formula I;
3) reacting said formula I ketone with methoxylamine or a salt thereof to give the compound of formula II; and
4) deprotecting said formula III compound in the presence of a base to yield the moxidectin product.

Alternatively, the compound of formula I may be deprotected to give the compound of formula IV and the formula IV compound may be reacted with methoxylamine or a salt thereof to give the desired moxidectin product. Accordingly, the invention also provides a process for the manufacture of moxidectin which comprises the following steps:
1) protecting the 5-hydroxy group of LL-F28249-α to give the compound of formula II;
2) reacting said formula II compound with stabilised o-iodoxybenzoic acid optionally in the presence of a solvent to give the ketone of formula I;
3) deprotecting said formula I ketone in the presence of a base to give the compound of formula IV; and
4) reacting said formula IV compound with methoxylamine or a salt thereof to yield the moxidectin product.
   The processes of the invention are shown in flow diagram II wherein R is a protecting group as defined hereinabove.

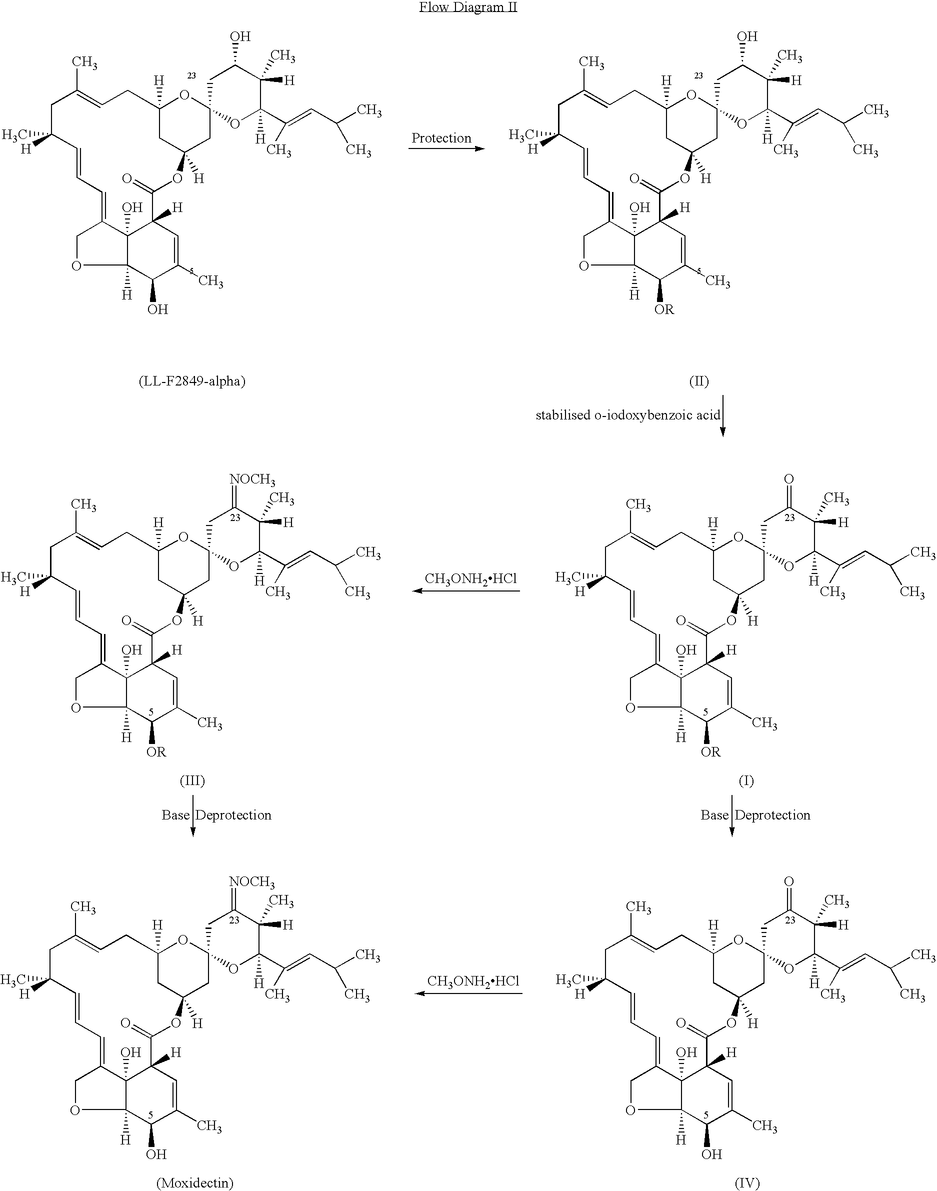

In actual practice, protection of the 5-hydroxy group of LL-F28249-α is achieved by the reaction of LL-F28249-α with a halide precursor of a protecting group as described hereinabove, for example p-nitrobenzoyl chloride, trimethylsilyl chloride, methoxymethylbromide, or the like, preferably p-nitrobenzoyl chloride, in the presence of an organic solvent such as toluene, methylene chloride, ethyl acetate, acetonitrile, or the like, preferably toluene, and an organic base such as pyridine, triethylamine, N-methylpyrrolidinone, or the like, preferably triethylamine. Oxidation of the protected LL-F28249-α compound of formula II is successfully achieved using the improved oxidation process described hereinabove, i.e. reacting said formula II compound with stabilised o-iodoxybenzoic acid optionally in the presence of a solvent to give the ketone of formula I. The formula I compound (either isolated and purified or as a solution of the crude reaction product in an organic solvent, such as toluene) is reacted with an aqueous solution of methoxylamine or a salt thereof and sodium acetate to give the protected moxidectin compound of formula III. Deprotection is achieved by reacting a solution of said formula III compound in an organic solvent such as toluene, dioxane, n-butanol or the like, preferably dioxane, with an aqueous solution of sodium hydroxide at 0°-25° C. and isolating the desired moxidectin product from the organic phase using standard procedures such as concentration and filtration or removal of the solvent.

In order to facilitate a further understanding of the invention, the following examples are presented primarily for the purpose of illustrating more specific details thereof. The invention is not to be limited thereby except as defined in the claims.

Unless otherwise noted, all parts are parts by weight. Stabilized o-iodoxy-benzoic acid (SIBX) was supplied by Simafex Company, France. The composition of the SIBX used was: 49% o-iodoxybenzoic acid; 29% isophthalic acid; and 22% benzoic acid. The terms HPLC and DMSO designate high performance liquid chromatography and dimethyl sulfoxide, respectively. In the chemical drawings, the term PNB designates p-nitrobenzoyl.

### EXAMPLE 1

### Preparation 5-O-(p-Nitrobenzoyl)-23-keto-LL-F28249-α

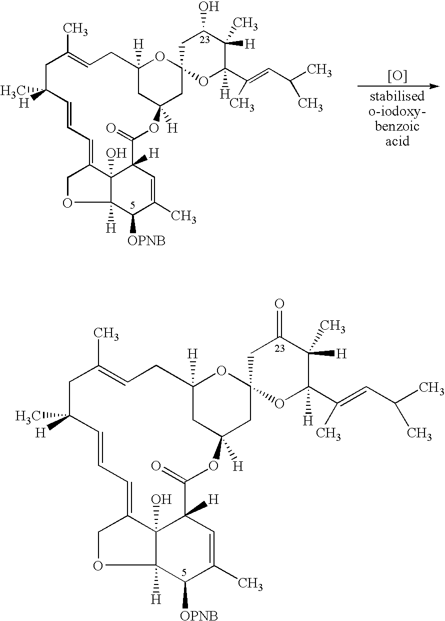

A solution of 5-O-(p-nitrobenzoyl)-LL-F28249-α (8.68 grams) in toluene was treated with a 20% w/w solution of SIBX (12 grams SIBX) in DMSO. The reaction mixture was stirred vigorously and maintained at 25° C. for 2 hours 30 minutes (92.7% conversion was obtained). The mixture was quenched with aqueous sodium sulfite solution (24% w/w concentration). The phases were separated and the toluene phase was analyzed by HPLC to give the title product in 88.4% yield.

### EXAMPLE 2

### Preparation 5-O-(p-Nitrobenzoyl)-23-keto-LL-F28249-α

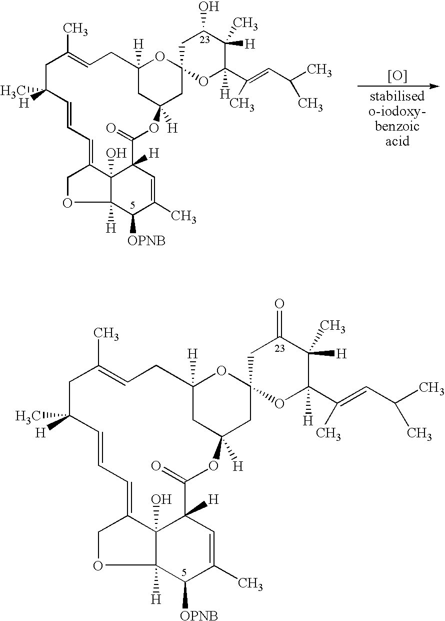

A solution of 5-O-(p-nitrobenzoyl)-LL-F28249-α (7.44 grams) in toluene was treated with a 30% w/w solution of SIBX (10.3 grams SIBX) in DMSO. The reaction mixture was stirred vigorously and maintained at 59° C. for 30 minutes (99.5% conversion was obtained). The mixture was quenched with aqueous sodium sulfite solution (24% w/w concentration). The phases were separated and the toluene phase was analyzed by HPLC to give the title product in 94.5% yield.

### EXAMPLE 3

### Preparation 5-O-(p-Nitrobenzoyl)-23-keto-LL-F28249-α

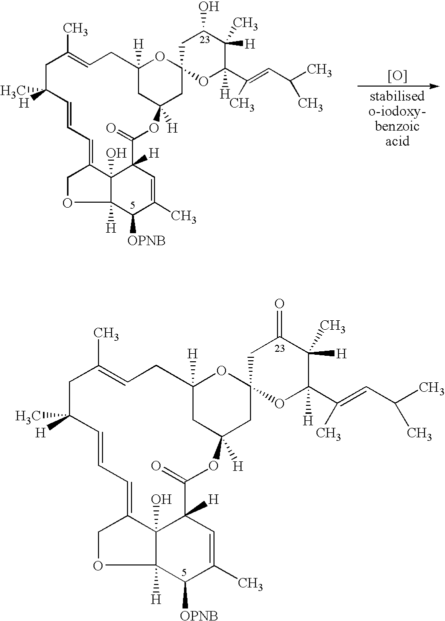

A solution of 5-O-(p-nitrobenzoyl)-LL-F28249-α (7.44 grams) in toluene was treated with a 30% w/w solution of SIBX (8.1 grams SIBX) in DMSO. The reaction mixture was stirred vigorously and maintained at 60° C. for 30 minutes (98.9% conversion was obtained). The mixture was quenched with aqueous sodium sulfite solution (24% w/w concentration). The phases were separated and the toluene phase was analyzed by HPLC to give the title product in 93.9% yield.

### EXAMPLE 4

### Preparation 5-O-(p-Nitrobenzoyl)-23-keto-LL-F28249-α

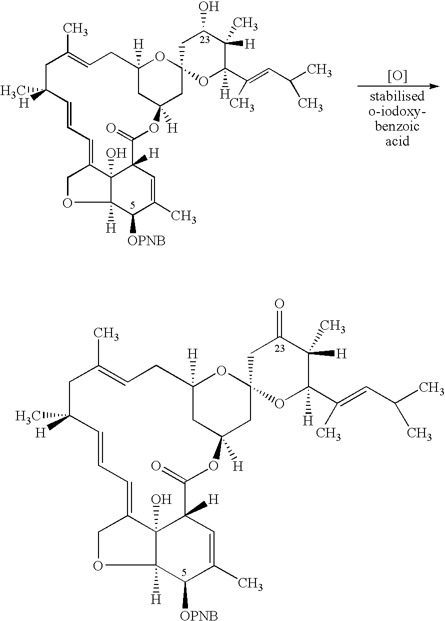

A solution of 5-O-(p-nitrobenzoyl)-LL-F28249-α (19.84 grams) in toluene was treated with 40.48 grams of DMSO, followed by treatment with solid SIBX (27.04 grams). The reaction mixture was stirred vigorously and maintained at 50° C. for 1 hour (98.5% conversion was obtained). The mixture was quenched with aqueous sodium sulfite solution (22% w/w concentration). The phases were separated and the toluene phase was analyzed by HPLC to give the title product in 88.1% yield.

What is claimed is:

1. A process for the selective oxidation of a 5-O-protected-LLF-28249-α compound of formula II 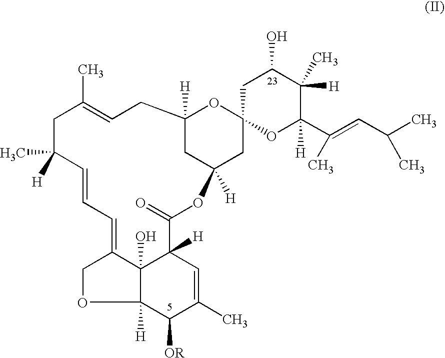
wherein R is a protecting group to the corresponding 23-keto compound of formula I 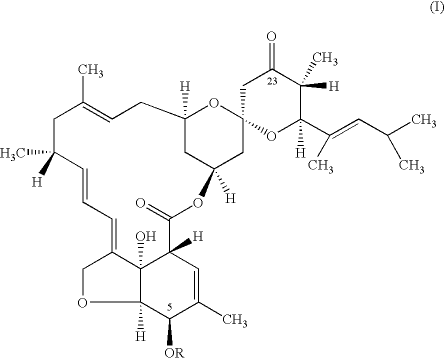
wherein R is as described for formula II which process comprises reacting said formula II compound with stabilised o-iodoxybenzoic acid, optionally in the presence of a solvent.

2. The process according to claim 1 wherein R is p-nitrobenzoyl.

3. The process according to claim 1 wherein said stabilised o-iodoxybenzoic acid is a mixture of o-iodoxybenzoic acid, isophthalic acid and benzoic acid.

4. The process according to claim 1 wherein the solvent is toluene.

5. The process according to claim 2 wherein the solvent is a mixture of toluene and dimethyl sulfoxide.

6. The process according to claim 2 wherein said stabilised o-iodoxy-benzoic acid is present in a wt/wt ratio of about 1.1 to 1.5 of o-iodoxybenzoic acid to 5-(p-nitrobenzoyl)-LL-F28249-α.

7. The process according to claim 3 wherein said mixture comprises about 48-50% of o-iodoxy-benzoic acid, about 28-30% of isophthalic acid and about 21-23% benzoic acid.

8. The process according to claim 7 wherein said mixture comprises about 49% of o-iodoxy-benzoic acid, about 29% of isophthalic acid and about 22% of benzoic acid.

9. The process according to claim 8 wherein said mixture is present at a wt/wt ratio of about 1.1 to 1.5 of o-iodoxybenzoic acid to 5-(p-nitrobenzoyl)-LL-F28249-α.

10. A process for the manufacture of moxidectin which comprises the following steps:
1) protecting the 5-hydroxy group of LL-F28249-α to give the compound of formula II 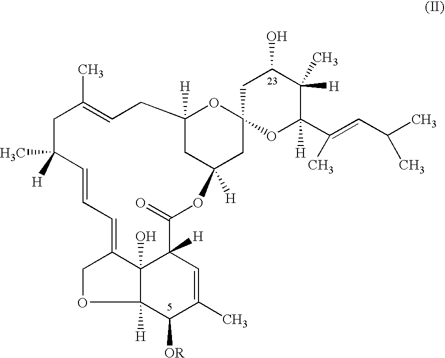
wherein R is a protecting group;
2) reacting said formula II compound with stabilised o-iodoxybenzoic acid optionally in the presence of a solvent to give the ketone of formula I 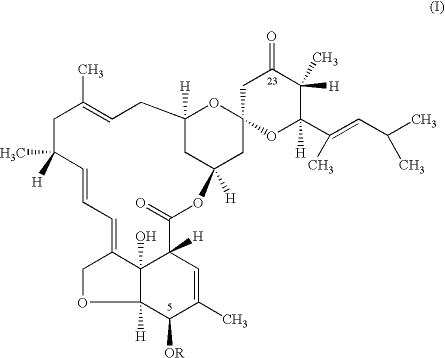
wherein R is as described for formula II;
3) reacting said formula I ketone with methoxylamine or a salt thereof to give the compound of formula III 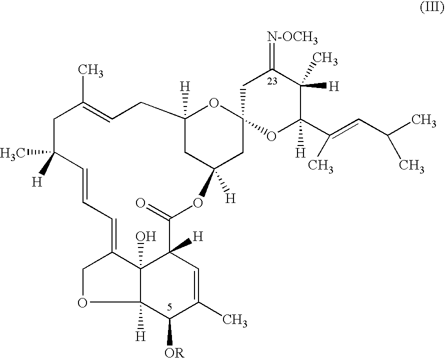
wherein R is as described for formula II; and
4) deprotecting said formula III compound in the presence of a base to yield the moxidectin product.

11. The process according to claim 10 wherein said stabilised o-iodoxybenzoic acid is a mixture of o-iodoxybenzoic acid, isophthalic acid and benzoic acid.

12. The process according to claim 11 wherein said mixture comprises about 48-50% of o-iodoxybenzoic acid, about 28-30% of isophthalic acid and about 21-23% benzoic acid.

13. The process according to claim 12 wherein said mixture comprises about 49% of o-iodoxy-benzoic acid, about 29% of isophthalic acid and about 22% of benzoic acid.

14. The process according to claim 13 wherein said mixture is present at a wt/wt ratio of about 1.1 to 1.5 of o-iodoxybenzoic acid to 5-(p-nitrobenzoyl)-LL-F28249-α.

15. A process for the manufacture of moxidectin which comprises the following steps:
1) protecting the 5-hydroxy group of LL-F28249-α to give the compound of formula II 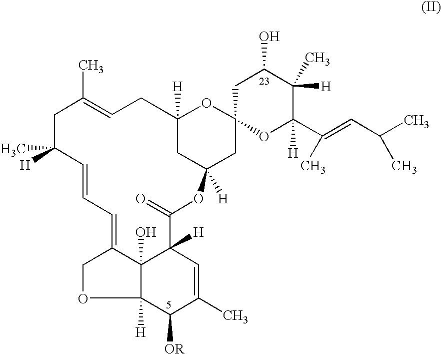
wherein R is a protecting group;
2) reacting said formula II compound with stabilised o-iodoxybenzoic acid optionally in the presence of a solvent to give the ketone of formula I 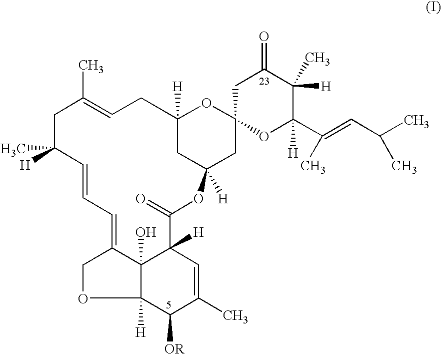
wherein R is as described for formula II;
3) deprotecting said formula I ketone in the presence of a base to give the compound of formula IV 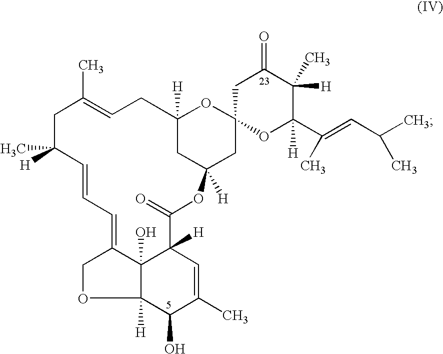
and
4) reacting said formula IV compound with methoxylamine or a salt thereof to yield the moxidectin product.

16. The process according to claim 15 wherein said stabilised O-iodoxybenzoic acid is a mixture of o-iodoxybenzoic acid, isophthalic acid and benzoic acid.

17. The process according to claim 16 wherein said mixture comprises about 48-50% of o-iodoxybenzoic acid, about 28-30% of isophthalic acid and about 21-23% benzoic acid.

18. The process according to claim 17 wherein said mixture comprises about 49% of o-iodoxy-benzoic acid, about 29% of isophthalic acid and about 22% of benzoic acid.

19. The process according to claim 18 wherein said mixture is present at a wt/wt ratio of about 1.1 to 1.5 of o-iodoxybenzoic acid to 5-(p-nitrobenzoyl)-LL-F28249-α.

20. The process according to claim 19 wherein the solvent is a mixture of toluene and dimethyl sulfoxide.
